(19) United States
(12) Patent Application Publication
(10) Pub. No.: US 2005/0148864 A1
(43) Pub. Date: Jul. 7, 2005

(54) Method and assembly for magnetic resonance imaging and catheter sterring

(76) Inventors: Robert Andrew Slade, Oxford (GB); Ian Leitch McDougall, Oxford (GB)

Correspondence Address: STAAS & HALSEY LLP, SUITE 700, 1201 NEW YORK AVENUE, N.W., WASHINGTON, DC, 20005, US

(21) Appl. No.: 10/509,847

(22) PCT Filed: Apr. 10, 2003
(86) PCT No.: PCT/GB03/01579

(30) Foreign Application Priority Data
Apr. 30, 2002 (GB) 0209892.9

(51) Int. Cl. A61B 5/05

(52) U.S. Cl. 600/423

(57) ABSTRACT

An imaging and catheter steering assembly comprising a magnetic field generating assembly (1-6) operable in a first mode to generate a first magnetic field in a working volume (7) located outside the assembly, the first magnetic field being suitable for use in a catheter, steering procedure. In a second mode the assembly (1-6) generates a second, static magnetic field in the working volume (7) suitable for conducting a magnetic resonance imaging process (MRI), the second magnetic field being more uniform in the working volume than the first magnetic field. A catheter (67) has a magnetic seed attached whose orientation, and hence the steering direction of the catheter, is determined by interaction with the first magnetic field.

Nuclear Magnetic Resonance (NMR) and specifically Magnetic Resonance Imaging (MRI) is the established imaging method of choice for many types of clinical diagnosis due to its exemplary soft tissue definition. Conventional whole body imaging systems generally use a superconducting solenoid or Helmholtz-type coil pair (“C Magnet”) to generate the required strong and uniform static magnetic field (called the B0 field). Patients undergoing examination lie within the bore of the solenoid or between the poles of the C-magnet. It is becoming increasingly desirable to monitor the progress and results of surgical procedures, such as biopsy and intravascular catheterisation using MR imaging. This process is often called interventional MRI, or I-MRI. Surgeons have restricted access to patients within conventional MRI apparatus, particularly solenoid magnets; this has hampered the widespread application of I-MRI techniques.

Many surgical procedures previously requiring large scale opening of the body cavity or brain-case are now routinely carried out using “keyhole surgery”. In this technique, often termed minimally invasive surgery (MIS), an endoscopic instrument or catheter is inserted through a small incision and remotely operated by a highly skilled surgeon. A related area is the new field of magnetically guided MIS, in which strong magnetic fields are used to manipulate and guide surgical instruments within the body. In particular, there have been recent advances in apparatus to remotely guide a catheter through a patient's vascular system by applying an external magnetic field with user-oriented flux direction. This field induces a torque on a permanent magnet “seed” implanted in the tip of a catheter, and thereby orients the tip in the desired direction. When the tip is correctly aligned, forward motion is provided by the surgeon pushing on the catheter from outside. In this way a catheter inserted at the groin can be guided into the heart or brain, obviating the need for traumatic opening of the chest or brain-case. The external magnetic field is generated by a set of three (or more) orthogonal superconducting coils. An example of suitable apparatus is described in U.S. Pat. No. 6,241,671. Clearly this apparatus limits direct access to the patient in the similar way to conventional MRI apparatus. (Note that the magnetic field generated by the apparatus of U.S. Pat. No. 6,241,671 is dominated by large gradients and is therefore entirely unsuitable for MR imaging).

A further related technology is monitoring the position of catheters within the patient's body using imaging. Commonly used techniques are X-ray fluoroscopy and ultrasound. X-ray fluoroscopy is particularly suitable for real-time imaging, as the catheter material has quite different X-ray absorption to tissue and is readily apparent in the images. Monitoring catheter position using MRI is more difficult because the catheter generates no measurable MR signal (only NMR signals from liquid sources are measured by conventional MRI hardware), and is therefore only visible by its contrast when immersed in tissue generating high NMR signal. Furthermore, whilst video-rate MR images are possible, they demand high-specification hardware, so real time catheter tracking using MRI is difficult. However, MRI has several advantages for tissue imaging compared to X-ray (see below) and it is often only necessary to take “snap-shots” of catheter position at certain critical stages of the surgical operation. Therefore many methods have been suggested to monitor catheter positions using MRI. These include using the susceptibility artefact created by the catheter to make it visible (i.e.: detecting local distortion of the B0 field) (for example U.S. Pat. No. 6,332,088, and C. J. G. Bakker, R. M. Hoogeveen, J. Weber, J. J. van Vaals, M. A. Viergever, W. P. Th. M. Mali, “MR-guided endovascular interventions: susceptibility-based catheter and near real-time scan technique”, Radiology 202, 273-276, 1996), and embedding tuned RF coils in the catheter tip (eg: U.S. Pat. No. 6,289,233).

As previously mentioned, MRI is often the preferred method for monitoring the progress of surgery compared to X-ray and ultrasound. There are several reasons for this, including: no ionising radiation (so theatre staff do not have to wear protective heavy lead clothing, which is particularly important for intricate brain or cardiac operations which may last several hours); MRI generates an undistorted 3D image (rather than a projection, with no depth information in the case of X-ray); MRI allows far better soft tissue characterisation and differentiation; MRI methods exist to monitor changes in tissue integrity, based on diffusion, perfusion and/or flow: for example MRI techniques exist to monitor temperature, (which is particularly useful during thermal tissue ablation or cryosurgery), cell populations and cell chemistry; subsequent MR images directly show changes caused by inflammation, internal bleeding, thrombosis, organ motion or the direct results of surgery, etc.; MRI contrast agents can also be used to highlight tissue changes (for example, this can be used to confirm that all of a malignant tumour has been removed before completing the operation). In specialised cases useful spectroscopic information can also be obtained from the MR image.

Significant efforts have been made to combine various benefits afforded by these technologies. For example the steering system of U.S. Pat. No. 6,241,671 incorporates X-ray fluoroscopy hardware (modified for use in strong magnetic fields) to monitor the catheter position in real-time. The Philips XMR system features a largely conventional MRI solenoid for tissue imaging and an X-ray fluoroscopy system for catheter position monitoring: the patient is placed on a moveable table that can be slid on rails between the two systems. In this way spatial registration of the MR and X-ray images is maintained. The latter system has the advantage over the first of providing high quality MR images when required, but it does not provide a catheter steering facility. Furthermore, it is rather cumbersome and clearly less than ideal to move the patient between the two systems when carrying out a delicate lengthy operation with the patient connected to life support and monitoring systems.

Both of these combined systems use separate hardware for the tasks of imaging the tissues, imaging the catheter and steering the catheter.

WO-A-99/18852 discloses the use of an MRI system for steering a catheter. The MRI system comprises a standard solenoid within which a working volume is defined while the catheter is provided with a set of orthogonal coils which can be selectively activated by the user so as to interact with the magnetic field generated by the solenoid to cause the orientation of the tip of the catheter to change. In some cases, the catheter tip is provided with a RF receiver and transmitter.

Although this system overcomes some of the problems of the prior art mentioned above it still requires a relatively complex steering apparatus to be provided on the catheter tip, and is furthermore not well suited to allow easy access of a surgeon to a patient located within the working region of a conventional closed MRI magnet, being a solenoid or C-magnet.

In accordance with the present invention, a catheter imaging and steering assembly comprises a magnetic field generating assembly operable in a first mode to generate a first magnetic field in a working volume located outside the assembly, the first magnetic field being suitable for use in a catheter steering procedure, and in a second mode to generate a second, static magnetic field in the working volume suitable for conducting a magnetic resonance imaging process (MRI), the second magnetic field being weaker but more uniform in the working volume than the first magnetic field; and a catheter having a magnetic seed attached whose orientation, and hence the steering direction of the catheter, is determined by interaction with the first magnetic field.

We have devised a new form of imaging and catheter steering assembly which has the advantage of utilizing the same magnetic field generating assembly for both imaging and steering, but has the added advantages of utilizing a working volume located outside the assembly and a simpler “magnetic seed” attached to the catheter. This latter facility reduces the complexity of hardware which needs to be provided at the catheter tip.

Consequently, the invention provides an assembly that combines the facility for MR imaging of tissues when required (e.g.: to evaluate the results of surgical intervention), with the ability to locate the catheter (preferably in the same images, thus obviating the need for later fusion of images from two different pieces of hardware, with the possibility for spatial mis-registration), while also allowing remote magnetic guidance of the catheter when required within the imaging volume. In addition, it would be most preferred that the apparatus allowed surgeons free and uninhibited access to the patient, and that the patient should not have to be moved during surgery.

In the steering mode the assembly provides a “vector rotate magnetic field” which is used to steer a catheter tip equipped with a magnetic seed. A “vector rotate magnetic field” is the term used for a magnetic field projected by a system of three (or more) electromagnets to cover a remote working region where steering is to take place. Typically the region is roughly spherical and sufficiently large to cover a significant proportion of the patient's body (e.g.: a 40 cm diameter sphere). Within this region the field has sufficient flux density to induce enough torque in a suitably sized “magnetic seed” to cause it to rotate within the body, overcoming the friction and obstruction forces from nearby body fluids and tissue structures, (about 0.2 T is sufficient, as will be explained later). The direction of the field can be selected over 4π solid radians by adjusting the currents in the three electromagnets, each of which generates a field component along one orthogonal axis (see U.S. Pat. No. 6,241,671). In all prior art steering systems the magnetic field within the imaging volume is unsuitable for imaging, being far too inhomogeneous.

The “magnetic seed” can be a simple, passive magnetic element or an active element whose magnetization can be locally controlled.

Examples of apparatus relevant to this invention can be found in WO 02/49705, WO 02/43797, and WO 02/56047.

An example of an imaging and catheter steering assembly according to the invention will now be described with reference to the accompanying drawings, in which:—

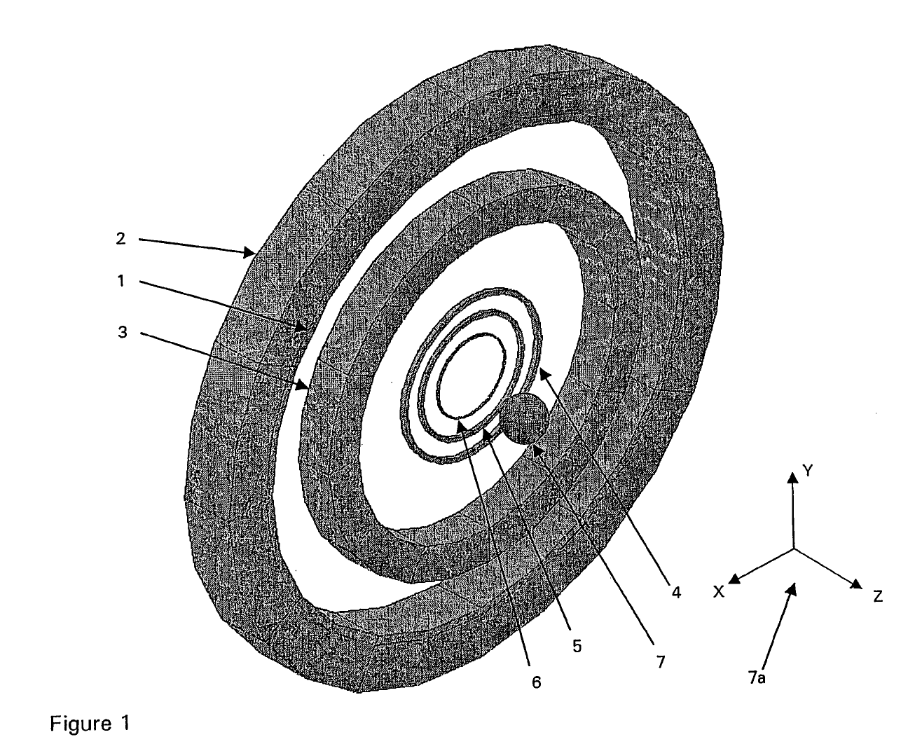

FIG. 1 is a schematic perspective view of a one-sided imaging magnet;

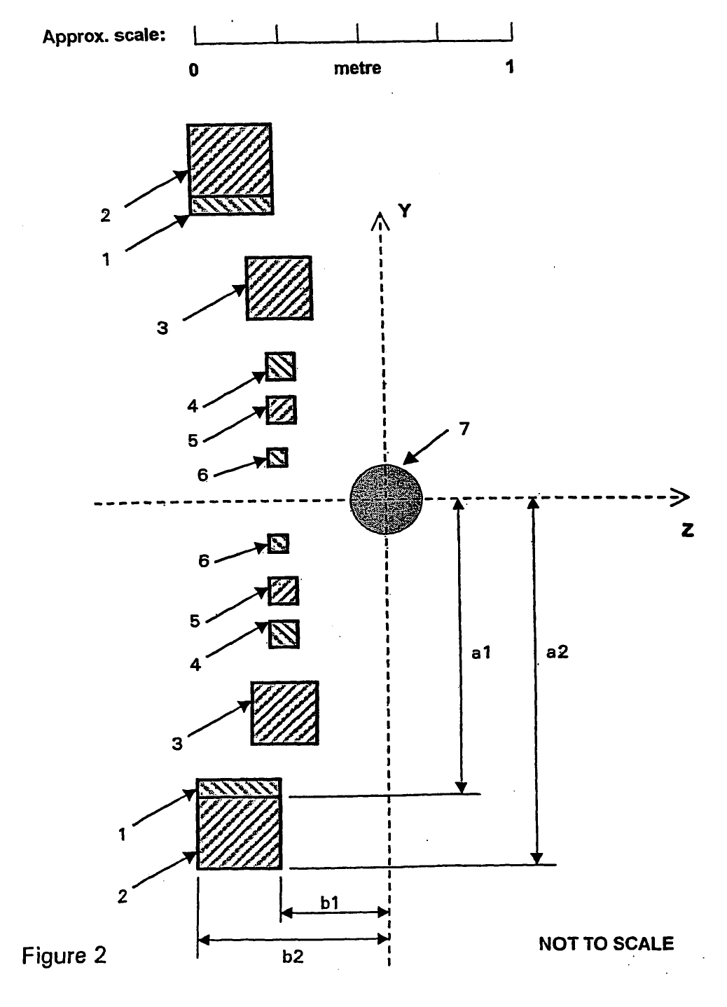

FIG. 2 is a schematic cross-section of the magnet, showing positions of the coils;

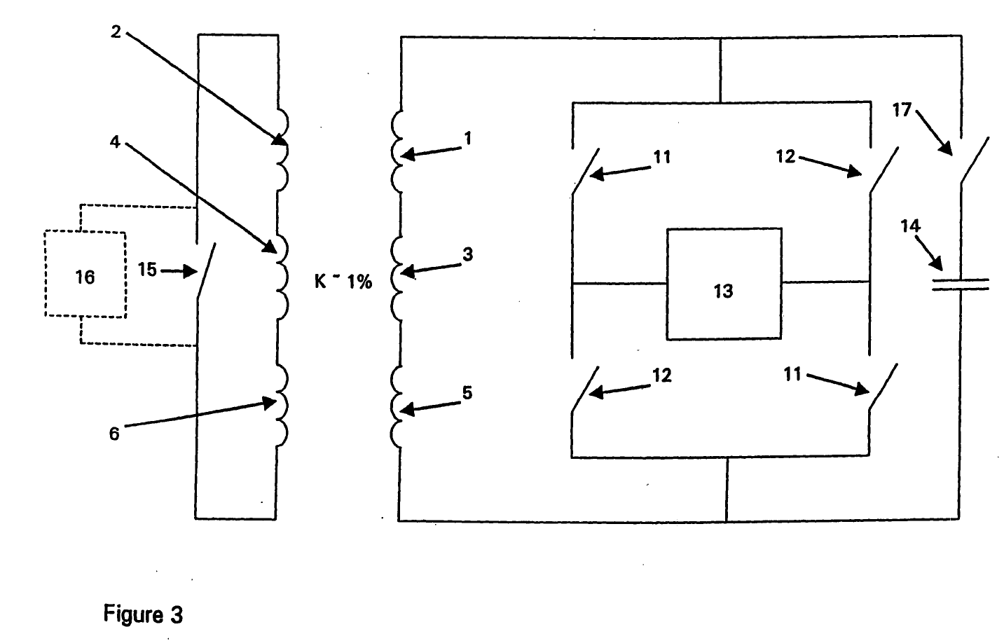

FIG. 3 is an example of suitable magnet circuit;

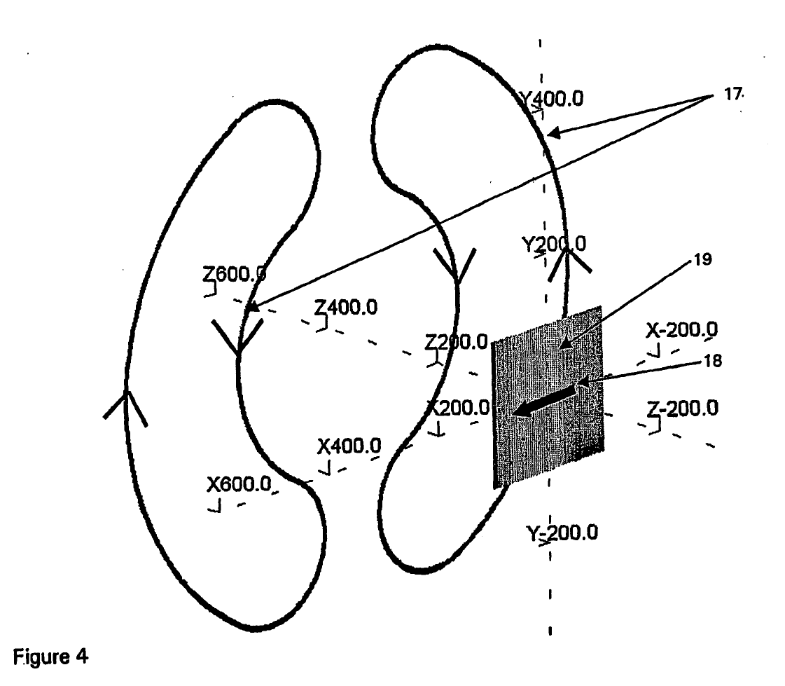

FIG. 4 is a perspective view of X axis gradient coil for imaging in the plane of the magnet;

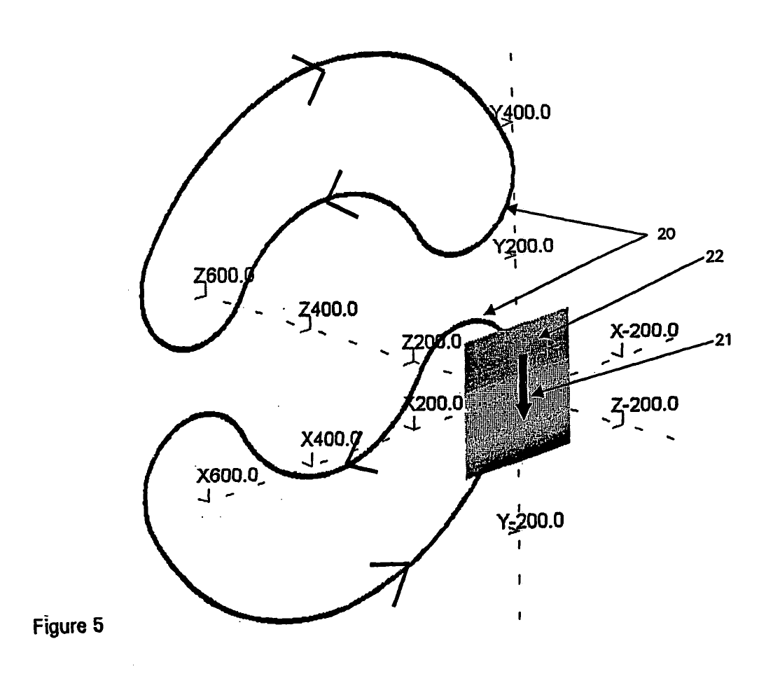

FIG. 5 is a perspective view of Y axis gradient coil for imaging in the plane of the magnet;

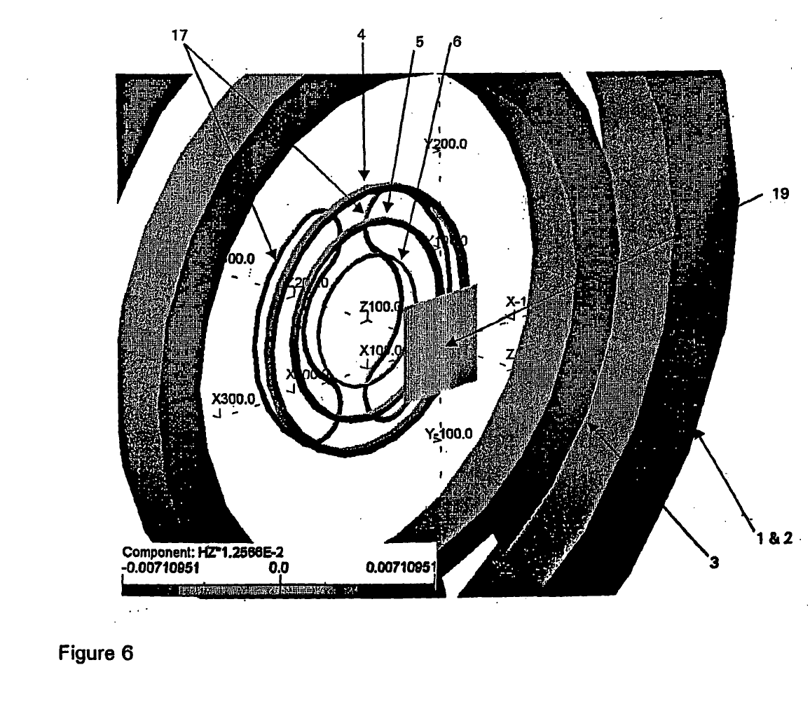

FIG. 6 shows the position of the X axis gradient coil relative to main magnet in perspective view;

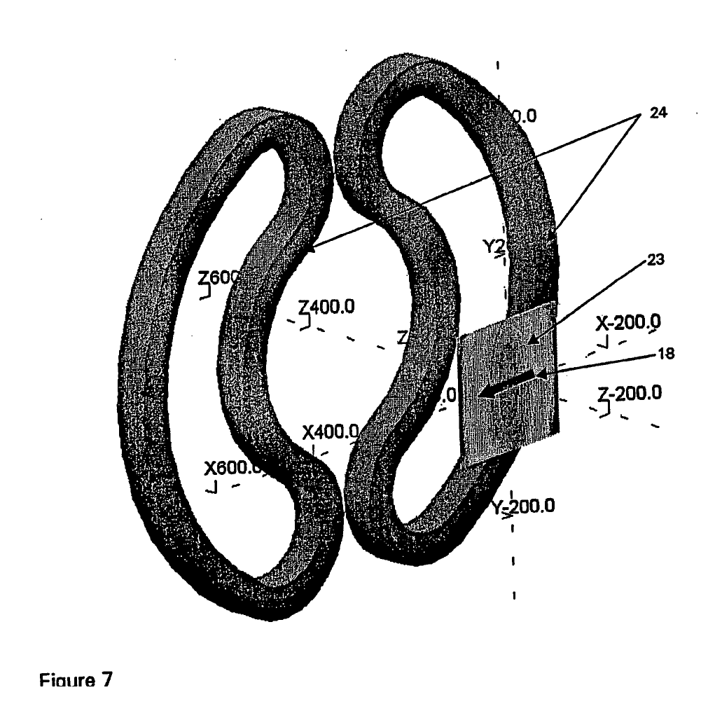

FIG. 7 is a perspective view of a modified X axis gradient and steering coil of the preferred embodiment;

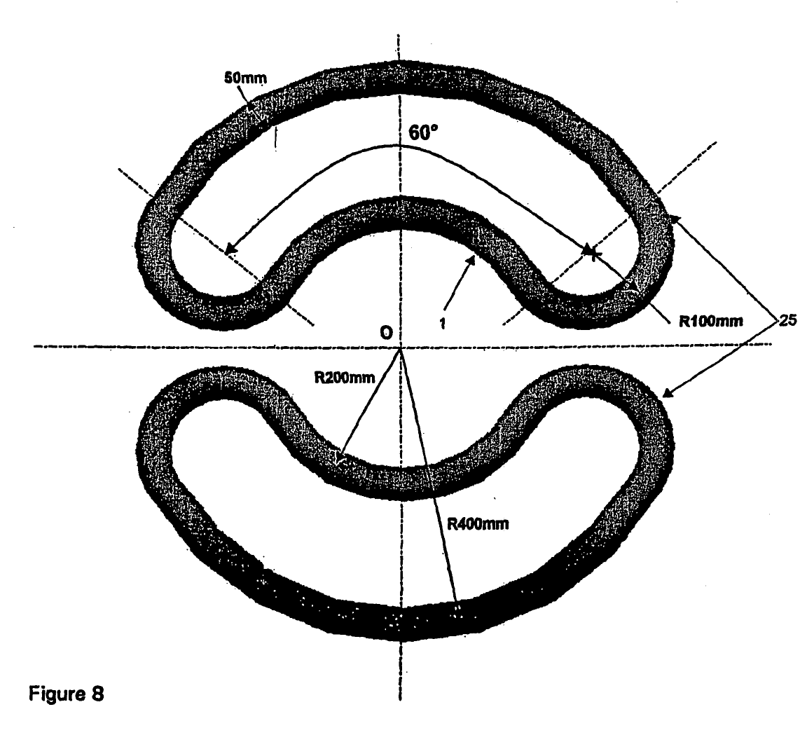

FIG. 8 shows dimensions of a modified Y axis gradient and steering coil of the preferred embodiment;

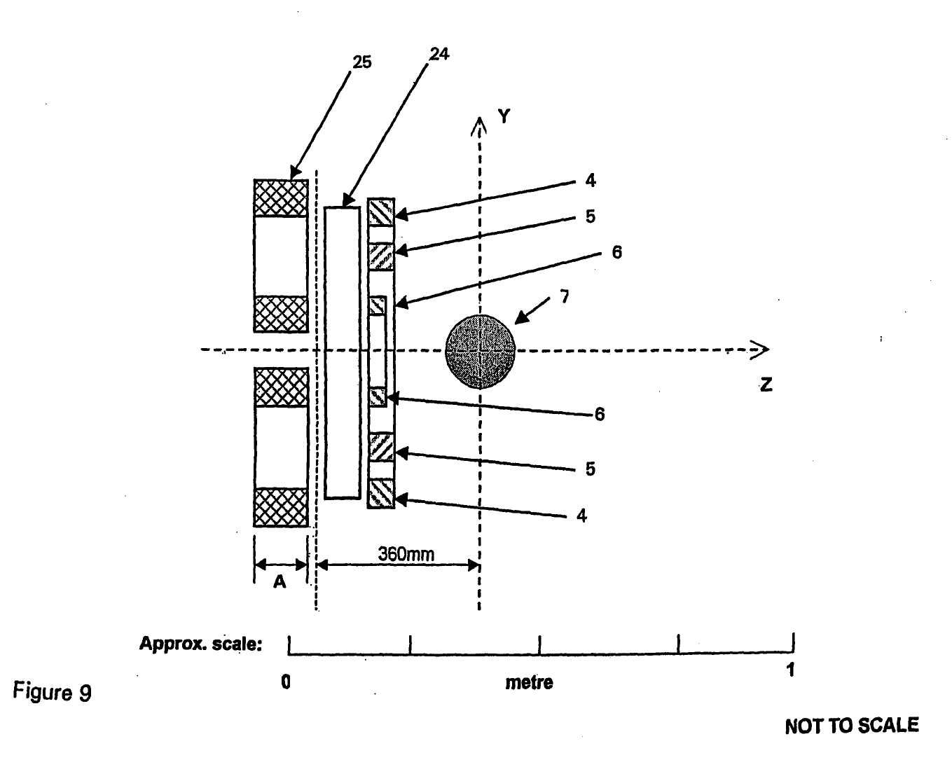

FIG. 9 is a cross-sectional view showing positions of main magnet coils and modified gradient/steering coils;

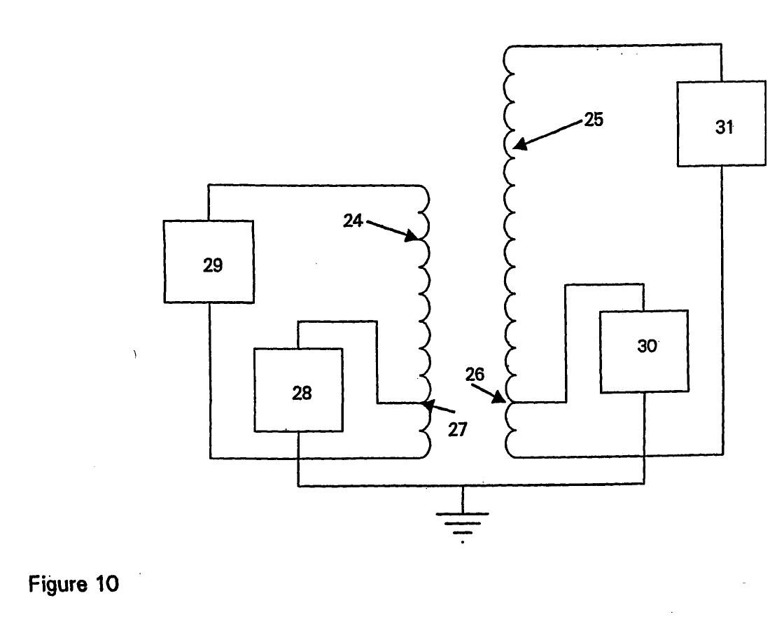

FIG. 10 is an example of a suitable circuit for combined X, Y steering and gradient coils;

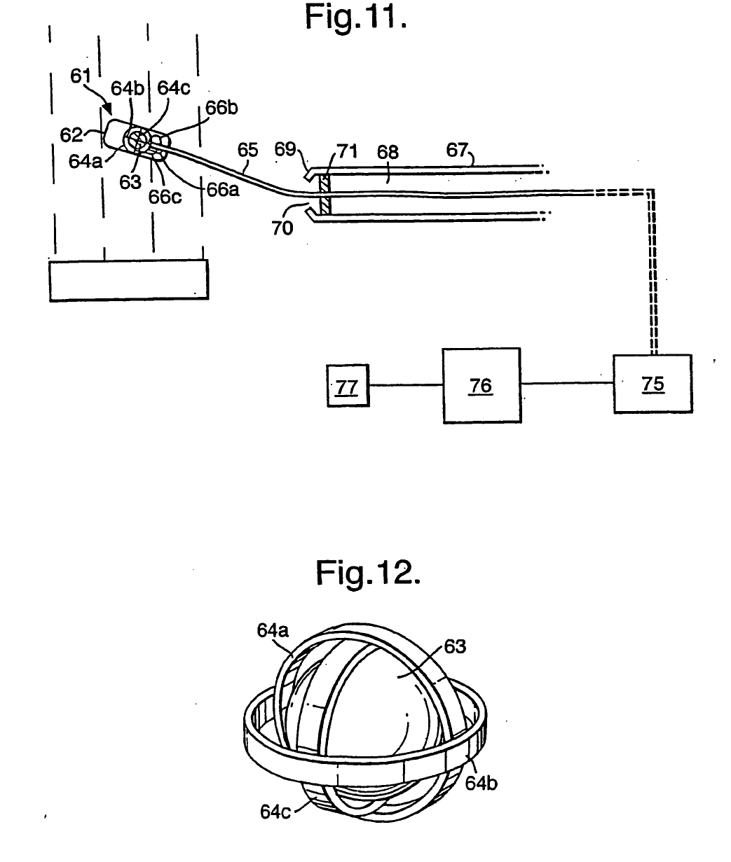

FIG. 11 is a schematic representation of a catheter steering system; and,

FIG. 12 is an illustration of the catheter steering assembly in more detail.

The present invention describes an arrangement of electromagnets, (preferably wound from high-temperature superconducting wire, optimised for high rate of change of current, ie: “high dI/dt” or “AC capable wire” and placed within a suitable cryostat (not shown) in which the currents can be adjusted under user control to allow MR images to be acquired from a remote sample volume in a second mode of operation, and to orient a magnetic seed embedded in a catheter tip within substantially the same volume for the purpose of steering the catheter in a first mode.

In the following description a preferred embodiment is described. It is not intended to limit the application to this embodiment, which is amenable to scaling and other geometry changes.

FIG. 1 shows an arrangement of co-planar electromagnet coils (about 3 m in diameter) for generating a 14 cm diameter uniform spherical working volume (DSV, 7) with flux density 0.1 T, uniform to 50 ppm. The DSV 7 is displaced by 21 cm to one side of the coil. MR imaging is possible within the DSV, using suitable gradient and RF fields, as will be described shortly. The Cartesian co-ordinate system (7a) is used throughout the description, and has its origin at the centre of the DSV. The coil positions, dimensions and currents required for imaging read-out mode are given in Table 1, which should be read with reference to FIG. 2 (showing the coil positions in cross-section).

TABLE 1
Flat 0.1T magnet for one-sided imaging system
(current directions shown for read-out mode).

| | | | | | | Current |
|---|---|---|---|---|---|---|
| Coil # | a1 (cm) | a2 (cm) | b1 (cm) | b2 (cm) | Turns | (A) |
| 1 | 121.18 | 125.28 | 27.18 | 52.82 | 1051 | 100 |
| 2 | 125.28 | 146.82 | 27.18 | 52.82 | 5523 | 100 |
| 3 | 76.24 | 93.77 | 21.24 | 38.77 | 3073 | −100 |
| 4 | 38.86 | 41.14 | 28.86 | 31.14 | 520 | 100 |
| 5 | 29.08 | 30.92 | 29.08 | 30.92 | 339 | −100 |
| 6 | 19.64 | 20.36 | 29.64 | 30.36 | 52 | 100 |

The flat electromagnet is composed of two distinct coil sub-circuits (or sub-magnets), one set comprising coils 1, 3 and 5, and one comprising coils 2, 4, and 6. (FIG. 3). Thus, as shown in FIG. 3, superconducting coils 2, 4, and 6 are connected in series to form the constant-field sub-magnet. After energisation by a DC power supply 16, a superconducting switch 15 is closed and the power supply 16 may be removed. Current continues to flow in the constant field sub-magnet. A second DC power supply 13 is connected to the variable-field sub-magnet (coils 1, 3, and 5) by an H-bridge inverter (switch pairs 11 and 12). These switches may be solid state devices, such as IGBTs or relays. When switch pair 11 are closed current flows in the variable-field sub-magnet, generating the pre-polarization field. The current in the constant field sub-magnet is unaffected due to the low coupling (coupling coefficient K^{˜}1%) between the magnets. After allowing a sufficient duration for the magnetization of the sample to build up, switches 11 are opened. The energy stored by the coils 1, 3 and 5 keeps the current flowing and charges a capacitor 14. The value of this capacitor is chosen so that it resonates with the self inductance of the variable field sub-magnet at the frequency defined by 1/TS, where TS is the desired field-switching time. For example, in the preferred 0.1 T magnet, the inductance of the variable-field sub-magnet is 21.5 Henries. For a switching time of 100 ms the capacitor 214 needs to be 47 uF. When the current has fallen to zero (the stored energy has exchanged from the magnet to the capacitor), the switches 12 are closed. Current then increases through the variable field sub-magnet in the opposite direction until it reaches the same level as before, but flowing in the opposite direction. Most of the energy is supplied from the capacitor, with the power supply making up any small losses. Switch 17 is then opened (the current flowing through it having fallen to zero). After applying the imaging pulse sequence, the current is again reversed by closing switch 17, opening switch pair 12, waiting until the current through the coils reaches zero, closing switch pair 11, waiting until the current through the coils reaches the peak value, then opening switch 17. This regenerative switching process may be repeated as often as required. Power supply 13 only needs to supply a small quantity of power to make up for any losses.

It is not a strict requirement to reverse the current to the same absolute level when flowing in the opposite direction—the correction coils could simply be turned off, or the current simply reduced. The former option would reduce the stored energy to 0.7 MJ during the pre-magnetization period, but there would only a 2.7 fold enhancement in field and NMR signal. However, there are good technical reasons for preferring to have the current rise to the same absolute level but flowing in the opposite sense in the pre-polarization period compared to the read-out period: the stored energy in both pre-polarization and read-out modes with reversed but equal-magnitude current is the same, so choosing any lower current value means that energy will have to be removed, stored for the duration of the imaging sequence, which could be a few seconds, then added back into the magnet. This is inconvenient: 0.1 MJ of energy would have to be stored in the example above. Resonant switching of the current using a capacitor, as described above, is the most convenient way of rapidly reversing the current to the same value, and temporarily storing the energy in the capacitor bank during the short change-over period TS. This is described in more detail in WO 02/56047.

The coil positions and turns-densities are arranged so that there is substantially zero flux linkage between the two sub-magnets. This means that the sub-magnets have substantially zero mutual inductance, a necessary requirement if they are to be treated as electrically isolated from the perspective of electronics design. However, with increased complexity in the electronics it is conceivable that sub-magnets with non-zero coupling could be used.

The present invention uses the same hardware to provide the steering vector rotate field for catheter steering. In particular, the main magnet provides the variable Z-component, and the X and Y gradient coils can provide the X and Y components of the vector rotate field.

The steering ability arises from the magnetic seed experiencing a torque trying to align it with the applied field. In steering mode gradients in the magnetic field within the DSV also undesirably impose translational forces on the seed, but these are insignificant compared to the twisting torque.

The torque on a small permanent magnet in a catheter tip having magnetic moment m in a magnetic field with flux density B is:
Γ=qm×B

The magnetic moment m of a cylindrical seed of permanent magnetic material with remanence B_{seed}, radius r and length l is:

$$m = l π {r}^{2} \frac{{B}_{seed}}{{μ}_{0}}$$

Experiment shows that the torque needed to deflect the tip is typically of the order 10 gramme centimetres (maximum torque occurs when the seed magnetisation is orthogonal to the applied magnetic field). Assuming typical values for the magnetic seed of r=1 mm, l=5 mm and B_{seed}=0.4 T (typical for hard ferrite), the required steering flux density is found to be 0.2 T. A hard ferrite seed can be de-magnetised by applying an oscillating decaying current to coil wound around the seed. The seed may be re-magnetised by a current pulse. This feature is described more fully below. It will be advantageous to use the ability to turn off the magnetic seed during imaging mode, and re-activate it for steering mode.

To generate the Z-component of the steering magnetic field vector from the main magnet coils with magnitude ranging from −0.2 T to +0.2 T it is necessary to adjust the coil currents to intermediate values compared to the currents needed in imaging mode. In comparison to imaging mode, the intermediate current values needed in steering mode require stored energy to be removed or added to the electromagnets using the power supplies. For example, the Z-component of steering field can be adjusted between −0.2 T to +0.2 T by setting the current in coils 2, 4 & 6 at the appropriate value between +74 amps and −74 amps, and turning off the current in coils 1, 3 & 5.

The maximum field gradient generated by the main magnets in steering mode occurs at either extreme of the −0.2 to +0.2 T range, and is 45 G/cm. The maximum force experienced by a magnetic seed of moment m in a magnetic field gradient is given by:
F=−m.grad(B)

For the seed in this example placed in the 45 G/cm gradient the maximum translational force is therefore 2.25 mN, which is negligible.

The X and Y components of the steering field are generated by flat X and Y gradient coils whose principles will be described with reference to FIGS. 4 to 6. These “kidney-shaped” gradient coils 17,20 lie in the plane of the magnet and generate linear dBz/dx and dBz/dy fields across an XY plane slice through the centre of the DSV. The fields generated by these coils also contain orthogonal field components, in the X and Y directions respectively. These orthogonal components have negligible effect on the MR image. FIG. 4 shows the X gradient coils (17), and FIG. 5 the Y gradient coils (20). The field orientation at the centre of the DSV is shown by the vectors (18 and 21). Also shown in FIGS. 4 and 5 are contour plots of the variation in the Z-component of the field generated by the coils across a 20×20 cm slice through the DSV (19 & 22), demonstrating the linearity of the gradient fields. The pair of kidney coils in each set are connected in series (not shown), with current orientation as shown by the arrows. FIG. 6 shows the X gradient coils, 17, in position relative to the main magnet coils, 1 to 6. The X gradient set is displaced by 360 mm along the Z axis and lies just behind the inner magnet coils (numbers 4, 5 & 6), as shown in FIG. 6. The Y gradient set (not shown) sits just behind the X gradient set.

To generate 0.2 T X and Y steering field components from these coils requires very much higher amp-turn values compared to operation as gradient coils. For example, to generate 0.2 T field requires about half a million amp-turns, in comparison to one thousand amp-turns needed to generate imaging gradient fields. Current densities this high cannot be achieved in reality with coils of the thin cross section shown in FIGS. 4 to 6. It is therefore necessary to increase the cross-sectional area of the coils. FIG. 7 shows a close up perspective view of the new “fatter” combined X-gradient and steering coils (24), and a contour plot of the X field component across the same 20×20 cm slice through the DSV (23). The variation in magnitude of the X component across the slice is only 15%, so the magnetic seed will experience only this variation in torque as its position within the slice varies. FIG. 8 shows the dimensions of the combined gradient and steering coil in the preferred embodiment. FIG. 9 is a cross-sectional view in the YZ plane showing the relative positions of the X and Y gradient/steering coils (24, 25) and the inner magnet coils (4, 5 & 6). The current density in each of the new gradient coils is about 200 amps/mm² and the winding cross sections are 50×50 mm for the X coil and 50×80 mm for the Y coil. The Y coil requires more amp-turns to generate the 0.2 T field because it is further from the DSV; it is therefore deeper than the X coil in the Z direction.

In practice to achieve the desired amp-turns without overheating, these coils must be made from superconducting wire, preferably high temperature superconductor (HTS). Second generation YBaCuO HTS wire is typically available as tape and is most easily wound into “pancake coils” (in which tape is wound upon itself to create a coil which is fat in the winding direction but only one tape-width thick). Assuming tape dimensions of 10 mm wide by 0.5 mm thick (including insulation) a 100 turn pancake coil would be 50 mm thick in winding direction and 10 mm wide. Therefore the X coil would require a stack of 5 pancakes connected in series and the Y coil a stack of 8. The total turns of the X coil is therefore 5×100=500 implying a coil current of 1000 amps is needed to achieve a 0.2 T field. This is achievable with a next generation HTS conductor.

FIG. 10 shows a possible circuit diagram for the X and Y gradient/steering coils. The physical layout of the coils results in near zero mutual inductance, so they can be treated as electromagnetically isolated units. The inductances 24 and 25 represent the total series inductance of the X and Y steering/gradient coils. These are connected to power supplies 29 and 31 which supply the necessary steady state current for steering mode. As previously described the coils are made from stacks of series connected pancake windings. The pancake windings are preferably connected together so that all coils lying in a plane are connected in series (ensuring current flows in the correct sense, as shown in FIGS. 4 & 5), then connected in series to the next layer. The coils are tapped (26 and 27) after the innermost pancake windings of the X and Y coils that face each other at z=360 mm. The taps are connected to independent power supplies 28 and 30 which supply the necessary fast rise time pulsed currents for gradient operation. In this way only the innermost pancakes of the stack nearest to z=360 mm are used for gradients. This offers best performance (the tapped coils have lower inductance coils allowing faster gradient pulse rise times, and the thinner cross section results in better gradient field linearity).

Clearly the superconducting gradient/steering coils will need to be cooled below their critical temperature. This can be achieved by placing them inside the magnet cryostat. The radio-frequency (RF) coils used for transmitting RF pulses and receiving NMR signals may be placed within the same cryostat, preferably in front of the inner magnet coils, close to the DSV. In this case the cryostat will require an RF transparent window. This is also described in more detail in WO 02/56047.

Any conventional imaging pulse sequence can be used.

As already explained, in addition to imaging, the assembly is used for steering a catheter. An example of a suitable catheter is shown in FIGS. 11 and 12. FIG. 11 shows a catheter steering assembly generally indicated at 61, the catheter steering assembly comprising a housing 62 within which is enclosed a sphere 63 of hard or semi-hard magnetic material such as ferrite, the sphere being enclosed by three orthogonal electric microcoils 64a,64b,64c.

A guide wire 65 is connected to the housing 62, the guide wire containing electrical lines 66a,66b,66c for supplying electric signals to the electrical coils 64a,64b,64c respectively. The guide wire 65 passes through a catheter generally indicated at 67, the catheter having an elongate body and a central bore 68 through which the guide wire passes.

At the end of the catheter body closest to the catheter steering assembly 61, an annular lip 69 is provided so as to narrow the diameter of the bore 68 to form an opening 70. At a predetermined distance along the guide wire from the catheter steering assembly 61, a disk 71 is attached to the guide wire 65, the radius of the disk being arranged to be just less than that of the internal diameter of the catheter 67 and yet larger than the diameter of the opening 70. During use, the attachment of the disk to the guide wire 65, prevents the catheter steering assembly 61 from separating from the catheter 67 by more than a predetermined distance. This distance can be arranged according to the use of the catheter in question.

The catheter guide assembly 61 and catheter 67, along with the guide wire 65, are formed from suitable materials to be used within the body of a living subject such as the human body. The guide wire 65 is lead out of the body and is adapted for manipulation by a surgeon. In this example the guide wire has sufficient stiffness to allow the catheter steering assembly 61 and catheter 67 to be moved through body cavities or lumens by applying a sufficient axial force to the guide wire 65.

The electrical lines 66a,66b,66c are attached to an external signal generator 75 which is adapted to provide electrical signals to the respective electrical lines 66a,66b,66c. The signal generator 75 is controlled by a computer 76 having a processor operating control software. An appropriate input device 77 such as a keyboard or joystick allows the surgeon to control the electrical signals being passed to the catheter steering assembly 61 using the computer 76.

The magnet of FIG. 1 is schematically represented at 78 and is positioned so as to apply a magnetic field with which the catheter steering assembly 61 may interact.

FIG. 12 shows the catheter steering assembly 61 in more detail, with the housing 62 removed. The ferrite sphere 63 is encircled by the three electric coils 64a,64b,64c. Each of these coils comprises a number of turns of high conductivity electrical wire, the coils being electrically connected to the electrical signal generator 75 using the corresponding electrical lines 66a,66b,66c positioned along the guide wire 65.

As indicated in FIG. 12, the three coils are arranged about the centre of the sphere 63 along mutually orthogonal axes. If sufficiently isotropic ferrite is used for the sphere 63, then the arrangement of the coils 64a,64b,64c in this manner allows the generation of a magnetic field within the ferrite in an arbitrary direction by superposition of the fields generated by each coil individually. This may be achieved by applying one or more suitable current pulses to one or more of the coils such that the combined magnetic field generated by the current in the coils is greater than the coercive force required to move the magnetic domains within the material.

In this manner, not only can the direction of the magnetisation be changed at will, the magnitude and polarity of this magnetisation can also be controlled. An additional benefit is that a decaying oscillating pulse of current applied to one or more of the microcoils can demagnetize the ferrite for subsequent MRI imaging.

1. An imaging and catheter steering assembly comprising: a magnetic field generating assembly operable in a first mode to generate a first magnetic field in a working volume located outside the assembly, the first magnetic field being suitable for use in a catheter steering procedure, and in a second mode to generate a second, static magnetic field in the working volume suitable for conducting a magnetic resonance imaging process (MRI), the second magnetic field being more uniform in the working volume than the first magnetic field; and a catheter having a magnetic seed attached whose orientation, and hence the steering direction of the catheter, is determined by interaction with the first magnetic field.

2. An assembly according to claim 1, wherein the magnetic field generating assembly comprises first and second electromagnets whose currents may be adjusted or reversed within a working range including zero, so as to vary the magnitude of the Z direction component of a vector rotate magnetic field in the working region as required in the steering procedure of the first mode, and may also be adjusted to a fixed setting which, in combination with the first and second electromagnet's coil positions and turns densities, generates a relatively uniform magnetic field in the working region as required in the imaging procedure of the second mode.

3. An assembly according to claim 2, further comprising third and fourth magnets for generating pulsed magnetic fields with linear gradients in mutually orthogonal X and Y directions respectively, orthogonal to the Z direction, during the MR process and substantially static magnetic fields in the X and Y directions respectively during the steering procedure.

4. An assembly according to claim 2, wherein one or more of the magnets are superconducting electromagnets.

5. An assembly according to claim 4, wherein the coils are made from high temperature superconductor.

6. An assembly according to claim 2, wherein the first and second magnets comprise electrical coils, the coil positions and turns densities being such that the coils exhibit substantially zero mutual inductance.

7. An assembly according to claim 1, wherein the magnetic dipole of the magnetic seed in the catheter tip may be switched on in steering mode and off in imaging mode.

8. An assembly according to claim 7, wherein the magnetic seed comprises a semi-hard permanent magnet whose magnetisation may be switched on by applying a pulse of current to a microcoil wound around the seed, and switched off by applying a decaying oscillating pulse of current to the same microcoil.

9. A method of carrying out a medical procedure, the method comprising: providing an imaging and catheter steering assembly comprising a magnetic field generating assembly operable in a first mode to generate a first magnetic field in a working volume located outside the assembly, the first magnetic field being suitable for use in a catheter steering procedure, and in a second mode to generate a second, static magnetic field in the working volume suitable for conducting a magnetic resonance imaging process (MRI), the second magnetic field being more uniform in the working volume than the first magnetic field; and a catheter having a magnetic seed attached whose orientation, and hence the steering direction of the catheter, is determined by interaction with the first magnetic field; inserting the catheter into a body; steering the catheter through the body by selectively operating the assembly in the first mode; and obtaining an image of part of the body by operating the assembly in the second, imaging mode.
